(19) United States
(12) Patent Application Publication
(10) Pub. No.: US 2012/0269781 A1
(43) Pub. Date: Oct. 25, 2012

(54) METHOD FOR PROLIFERATING HAIR FOLLICLE STEM CELLS

(75) Inventors: Jeong Chan Ra, Gyeonggi-do (KR); Sung Keun Kang, Seoul (KR); Sang Kyu Woo, Gyeonggi-do (KR); Mi Ae Kim, Gyeonggi-do (KR); Eun Ji Yoon, Gyeonggi-do (KR)

(73) Assignee: RNL BIO CO., LTD., Seoul (KR)

(21) Appl. No.: 13/505,127

(22) PCT Filed: Nov. 5, 2010
(86) PCT No.: PCT/KR2010/007794

§ 371 Date: Jul. 11, 2012

(30) Foreign Application Priority Data
Nov. 6, 2009 (KR) 102009107184

(51) Int. Cl. C12N 5/071; A61P 17/14; A61K 35/36

(52) U.S. Cl. 424/93.7; 435/377; 435/325

(57) ABSTRACT

The present invention relates to a method of proliferating follicular stem cells in high yield, and more particularly, to a method of proliferating follicular stem cells in large amounts by culturing the cells using a specific medium containing a specific concentration of a Rho-associated kinase (ROCK) inhibitor and to a medium which is used in the method.

### TECHNICAL FIELD

The present invention relates to a method of proliferating follicular stem cells in high yield, and more particularly, to a method of proliferating follicular stem cells in large amounts by culturing the cells using a specific medium containing a specific concentration of a Rho-associated kinase (ROCK) inhibitor, and to a medium which is used in the method.

### BACKGROUND ART

Recently, with an increasing interest in beauty, an interest in the treatment of alopecia has also increased. Alopecia refers to hair loss in areas of skin that normally have hair. Although hair does not have an important physiological function which is related directly to life, it plays a very significant role in terms of beauty and functions to block UV light and protect the head. Severe hair loss can have negative effects not only on social life, but also on mentality, and thus hair is important in terms of the quality of life.

Hair loss can be divided into two categories: scarring hair loss in which the skin scars; and non-scarring hair loss in which only hair falls out. In the case of scarring hair loss, a hair follicle is broken, and thus hair never regrows. Hair grows in follicles, and each follicle undergoes repeated cycles of growth and rest. The interval of the hair cycle varies depending on the parts of the body. The hair of the head undergoes a growth phase (anagen) of about 2-6 years, a regression phase (catagen) of about 2-4 weeks, and a resting phase (telogen) of about 3-4 months. Each follicle undergoes 10-20 hair follicle growth cycles during a person's lifetime.

Various methods for treating hair loss have been proposed, but in recent years, methods of treating hair loss using genes have received attention. For example, a therapeutic method of delivering a desired DNA code directly into a follicle using a gene structure or a therapeutic method of inhibiting gene expression has been developed. However, the efficacy, cost and safety of this therapeutic method and the effect of this method on future generations have not yet been completely established. Thus, even if a gene which is involved in hair loss is found, a significant amount of time is required until a method of treating hair loss using the gene is used with safety.

Meanwhile, a hair regeneration method was reported which comprises extracting hair in the growth phase in a state in which the hair bulb is attached to the hair, culturing the cells of the hair follicle, and transplanting the cultured cells into the site from which the hair was extracted. However, this method was so not effective.

Meanwhile, methods of treating hair loss using stem cells have been proposed. Such methods comprise identifying follicular stem cells and analyzing the genes of the cells and are expected to be used to treat hair loss or other skin diseases such as a burn. The concept of epidermal stem cells has already been discussed from 30 years ago. Hair growth is a unique cyclic regeneration phenomenon, and the location and function of hair follicular stem cells is a crucial issue for understanding both biology and pathology of hair growth [Oshima H. et al., Cell, 104:233-245, 2001]. Label-retaining cells, a characteristic of stem cells, were found to reside in the permanent upper portion of hair follicle, the so-called the bulge area [refer: Cotsarelis G. et al., Cell, 61:1329-1337, 1990].

However, a method for culturing such follicular stem cells has not yet been clearly established. Although it is known that follicular stem cells are present in some follicles, a large amount of stem cells are required for the practical treatment of human baldness, but technology of proliferating isolated stem cells as much as they can be clinically applied is still unsatisfactory. In addition, a marker protein for stem cells has not yet been clearly identified, and thus a method for treating hair loss using stem cells is still unsatisfactory.

Accordingly, the present inventors have made extensive efforts to produce large amounts of follicular stem cells, and as a result, have found that larger amounts of follicular stem cells can be proliferated and maintained in a specific kind of medium among various media containing a specific concentration of a Rho-associated kinase (ROCK) inhibitor, thereby completing the present invention.

### DISCLOSURE OF INVENTION

It is an object of the present invention to provide a medium for producing a large amount of follicular stem cells.

Another object of the present invention is to provide a method of using said medium to proliferate follicular stem cells in such large amounts that the follicular stem cells are clinically applicable.

Still another object of the present invention is to provide a composition for treating hair loss, which contains as an active ingredient a large amount of follicular stem cells obtained by said method.

To achieve the above objects, the present invention provides a medium for proliferating and maintaining follicular stem cells, which contains 5-50 μM of a Rho-associated kinase (ROCK) inhibitor in a basal medium. Herein, the basal medium may be one selected from the group consisting of an M199/F12 (mixture) medium, a MEM-alpha medium, a low-concentration glucose-containing DMEM medium, a MCDB 131 medium, and an IMEM medium. Herein, the low-concentration glucose-containing DMEM medium contains glucose at a concentration of about 1000 mg/L. Among the above media, the M199/F12 (mixture) medium or the MEM-alpha medium is particularly preferred.

The basal medium preferably contains an ITS+ (insulin, transferrin, selenium) premix, EGF, bFGF, and antibiotics, for example, an antibiotic/antimycotic mixture. Particularly, the concentration of the ROCK inhibitor is preferably 5-20 μM, and more preferably about 10 μM.

The present invention also provides a method for proliferating and maintaining follicular stem cells, the method comprising subculturing isolated follicular stem cells in a medium comprising a basal medium supplemented with 5-50 μM of a Rho-associated kinase (ROCK) inhibitor.

Herein, the isolated follicular stem cells are preferably subcultured to passage 2-6, and the medium is replaced at 2-day intervals during culture of the cells.

The present invention also provides a cell therapeutic composition for treating alopecia or atrichosis, which contains as an active ingredient a large amount of follicular stem cells obtained by said method. Herein, the composition may further contain adipose stem cells which are relatively easy to isolate.

### BRIEF DESCRIPTION OF THE DRAWINGS

FIGS. 1 to 5 are photographs showing follicular stem cells cultured in 9 kinds of media.

FIGS. 6 and 7 show a comparison of the proliferation ability of follicular stem cells between various concentrations of the ROCK inhibitor.

FIG. 8 is a comparison of the proliferation ability of follicular stem cells between a commercial M199/F12 medium composition and a composition containing ITS+ premix, EGF, bFGF and an antibiotic/antimycotic mixture in M199/F12 medium.

FIG. 9 is a set of graphs showing the results obtained by inducing hair loss in mice with dihydrotestosterone, administering each of saline, 3% minoxidil, follicular stem cells, adipose stem cells, and a combination of follicular stem cells and adipose stem cells, and measuring the condition of hair loss in the mice weekly at weeks 0-14.

FIG. 10 is a set of photographs showing the results obtained by inducing hair loss in mice with dihydrotestosterone, administering each of saline, 3% minoxidil, follicular stem cells, adipose stem cells, and a combination of follicular stem cells and adipose stem cells, and photographing the condition of hair loss in the mice weekly at weeks 6-14.

FIG. 11 is a set of graphs showing the results obtained by inducing hair loss in mice with testosterone, administering each of saline, 3% minoxidil, follicular stem cells, adipose stem cells, and a combination of follicular stem cells and adipose stem cells, and measuring the condition of hair loss in the mice weekly at weeks 0-16.

FIG. 12 is a set of photographs showing the results obtained by inducing hair loss in mice with testosterone, administering each of saline, 3% minoxidil, follicular stem cells, adipose stem cells, and a combination of follicular stem cells and adipose stem cells, and photographing the condition of hair loss in the mice weekly at weeks 6-16.

FIG. 13 is a set of photographs showing the results obtained by administering a combination of follicular stem cells and adipose stem cells and photographing the condition of hair loss in the mice weekly at weeks 0-6 (week 0: the time point of administration).

### BEST MODE FOR CARRYING OUT THE INVENTION

Hereinafter, the present invention will be described in detail.

Stem cells refer to cells having not only self-replicating ability but also the ability to differentiate into at least two types of cells.

Adult stem cells are stem cells that appear either in the stage in which each organ of an embryo is formed after the developmental process or in the adult stage. It is known that adult stem cells are multipotent and capable of differentiating into tissue- and organ-specific cells. Such multipotent stem cells, which are stem cells capable of differentiating into cells specific to tissues and organs containing these cells, are involved not only in the growth and development of various tissues and organs in the fetal, neonatal and adult periods but also in the maintenance of homeostasis of adult tissue and the function of inducing regeneration upon tissue damage.

The present invention is directed to the effective culture of adult stem cells, particularly follicular stem cells derived from epithelial tissue.

Follicular stem cells include about 10% of stem cells, which are located in the basal cell layer of epidermis, the so-called the interfollicular epidermis, and stem cells present in hair follicles. Particularly, follicular stem cells which are undifferentiated cells are known to play an important role in the regeneration of the epidermis.

The follicular stem cells which are used in the present invention may be isolated from the epidermal tissue (e.g., scalp tissue) of all mammals, including humans. Herein, the scalp tissue must include follicles.

Specifically, the expression “follicular stem cells derived from epidermal tissue” or “follicular stem cells derived from scalp tissue” refers to undifferentiated adult stem cells isolated from epidermal tissue and is also abbreviated herein as “follicular stem cells”. These follicular stem cells can be obtained by conventional methods known in the art. In one example of the present invention, follicular stem cells derived from human scalp tissue were used.

One example of the conventional methods known in the art may be the following method.

In order to isolate follicular stem cells from scalp tissue, scalp tissue is finely cut. Then, follicular tissue is isolated from the cut tissue. The isolated follicular tissue is finely cut and chemically degraded in collagenase-containing medium. Herein, the chemical degradation of the cut tissue can be performed in a gravity convection incubator at 50-200 rpm at 30˜40° C. for 0.5-24 hours. The chemically degraded tissue cells (scalp cells) are collected and cultured in serum-containing medium, and the cultured cells are collected and follicular stem cells are isolated therefrom.

In one aspect, the present invention is directed to a culture medium for proliferating and maintaining a large amount of follicular stem cells, which contains a Rho-associated kinase (ROCK) inhibitor in a basal medium.

The medium that is used for the proliferation of a large amount of follicular stem cells in the present invention may be a basal medium which is generally used for culture of cells. As used herein, the term “basal medium” refers to a medium having a simple composition, in which cells can proliferate. General examples of the basal medium that is used for cell culture in the present invention include MEM (Minimal Essential Medium), DMEM (Dulbecco modified Eagle Medium), RPMI (Roswell Park Memorial Institute Medium), and K-SFM (Keratinocyte Serum Free Medium), as well as other media which are used in the art.

Preferably, the basal medium may be selected from the group consisting of a M199/F12 mixture (GIBCO), a MEM-alpha medium (GIBCO), a low-concentration glucose-containing DMEM medium (Welgene), a MCDB 131 medium (Welgene) and an IMEM medium (GIBCO). Herein, the low-concentration glucose-containing DMEM medium contains glucose at a concentration of about 1000 mg/L. Among these media, the M199/F12 mixture (GIBCO) or the MEM-alpha medium (GIBCO) is preferably used.

Herein, the media preferably contain ITS+ premix (insulin, transferrin, selenious acid), EGF, and bFGF. In addition, the media may further contain antibiotics, for example, an antibiotic/antimycotic mixture.

The ITS+ premix is a culture supplement that contains insulin, human transferring and selenious acid, which are the essential elements of defined media, and it functions to stimulate the proliferation of cells under serum-free culture conditions. EGF (epidermal growth factor) binds to its receptor so as to regulate the growth, proliferation and differentiation of cells. bFGF (basic fibroblast growth factor) is a cell growth factor that is involved in angiogenesis, wound healing and the like and generally plays a pivotal role in the proliferation and differentiation of various types of cells.

Particularly, the media that are used in the present invention are effective for the culture and proliferation of follicular stem cells. Although a medium which is generally known to be used for culture of follicular stem cells is DMEM or K-SFM, specific media which are more effective for the culture of follicular stem cells were selected in Example 2 of the present invention.

In the culture of follicular stem cells, the concentration of each of insulin, transferrin and selenious acid is 0.1 ug/ml (100 ng/ml)-10 ug/ml, preferably 0.1-6.25 ug/ml, and more preferably 0.1-1 ug/ml. In one example of the present invention, each of insulin, transferrin and selenious acid was used at a concentration of about 0.625 ug/ml.

The media are characterized by containing 5-50 μM of a Rho-associated kinase (ROCK) inhibitor. The ROCK inhibitor is preferably contained at a concentration of 5-20 μM, more preferably 7-15 μM, and most preferably about 10 μM.

Isolated follicular stem cells are cultured in the medium supplemented with the ROCK inhibitor, and the cultured follicular stem cells are recovered. Then, the cells are suncultured in the presence of the ROCK inhibitor, whereby the follicular stem cells can be maintained in an undifferentiated state.

The Rho-associasted kinase (ROCK) inhibitor is a substance functioning to inhibit apoptosis and is known to function to inhibit agonist-induced Ca²⁺ sensitization in axon regeneration, myosin phosphorylation and smooth muscle contraction. More specifically, it is known that the ROCK inhibitor alleviates abnormalities in muscle cells that cause hypertension and asthma, and functions to increase blood flow in the optic nerve head and continuously reduce intraocular pressure. In addition, it is known to have the biological functions of inhibiting apoptosis and maintaining cells in an undifferentiated state.

Typical examples of the ROCK inhibitor that is used in the present invention include Y-27632, HA-1077, Y-39983, Wf-536 and the like. Among them, Y-27632 (Calbiochem or Sigma) was used in one example of the present invention. Y-27632 (Calbiochem or Sigma) has a structure represented by the following formula 1:

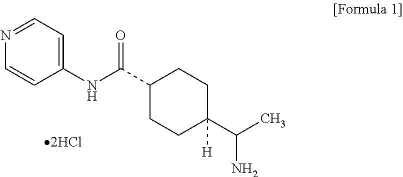

The concentration of the ROCK inhibitor which is used to treat follicular stem cells in the present invention is 5-50 μM, preferably 5-20 μM, and more preferably about 10 μM. If the ROCK inhibitor is used at a concentration of less than 5 μM, the undifferentiated state of follicular stem cells will be difficult to maintain for a long time, and if it is used at a concentration of more than 50 μM, cells can morphologically change and can enter the differentiation phase.

Particularly, when follicular stem cells are treated with the above-described concentration of the ROCCK inhibitor, the proliferated follicular stem cells will be maintained in a healthy state from morphological and functional viewpoints for a long time.

In another aspect, the present invention is directed to a method for proliferating and maintaining a large amount of follicular stem cells using the above medium. That is, the present invention is directed to a method for proliferating and maintaining follicular stem cells, the method comprising subculturing isolated follicular stem cells in a specific medium comprising a basal medium supplemented with a Rho-associated kinase (ROCK) inhibitor.

The above-described medium is also used in the inventive method for proliferating and maintaining follicular stem cells, and thus the detailed description thereof will be omitted.

Particularly, the isolated follicular stem cells are subcultured to passage 2-6, preferably to passage 2. In addition, the medium is replaced at 2-day intervals during culture of the cells. In order for follicular stem cells, which proliferated by subculture, to be effectively applied in clinical practice, the morphology and function of the cells should not easily change during the subculture period. A specific medium which contains a specific concentration of the ROCK inhibitor and specific components functions to prevent such morphological and functional changes from occurring.

In still another aspect, the present invention is directed to a cell therapeutic agent for inducing hair growth, an agent for treating alopecia, or an agent for treating atrichosis, which contains as an active ingredient a large amount of follicular stem cells obtained by said method. In addition, the present invention is directed to a method for treating alopecia or atrichosis using a large amount of follicular stem cells obtained by said method. Preferably, these compositions may contain not only follicular stem cells, but also adipose stem cells. Adipose stem cells may be obtained by any method known in the art. For example, adipose stem cells may be obtained by washing adipose tissue, degrading the washed tissue with, for example, collagenase, centrifuging the degraded tissue, removing the supernatant, and culturing the remaining adipose stem cells in a serum medium overnight or longer.

As used herein, the expression “induction of hair growth” refers to the ability to form a follicle in an area of hair loss or a hair-free area so as to induce hair growth.

Unless otherwise indicated, the term “treating”, as used herein, means reversing, alleviating, inhibiting the progress of, or preventing the disorder or condition to which such term applies, or one or more symptoms of such disorder or condition. The term “treatment”, as used herein, refers to the act of treating as “treating” is defined immediately above. Thus, as used herein, “treating” or “treatment” of a disease in a mammal includes one or more of:
(1) inhibiting growth of the disease;
(2) preventing spread of the disease;
(3) relieving the disease;
(4) preventing recurrence of the disease; and
(5) palliating symptoms of the disease.

In order to treat alopecia or atrichosis, the composition of the present invention is administered in a pharmaceutically effective amount.

As used herein, the term “therapeutically effective amount” means that an amount of the compound being administered alleviates to some extent one or more of the symptoms of the disease being treated. Therefore, the therapeutically effective amount refers to an amount that has the effect of: (1) reversing the rate of progress of a disease; (2) inhibiting the progress of the disease to some extent; or (3) relieving (preferably eliminating) one or more symptoms associated with the disease to some extent.

The inventive cell therapeutic agent for inducing hair growth, the inventive agent for treating alopecia and the inventive agent for treating atrichosis may preferably be administered by parenteral routes, including intravenous, intraabdominal, intramuscular, subcutaneous or topical routes. More preferably, these agents may be administered subcutaneously or topically and may be injected directly into an area of hair loss or an area in need of hair growth.

For injection, the composition of the present invention may preferably be formulated with a pharmacologically acceptable buffer, such as Hank's solution, Ringer's solution or physiological saline buffer. For transmucous administration, non-invasive agents suitable for a barrier through which the composition is to be passed are used in formulation. Such non-invasive agents are generally known in the art.

Formulations for parenteral administration include sterilized aqueous solutions, non-aqueous solvents, suspending agents, emulsifying agents. Suspending agents and emulsifying agents that may be used in the present invention include vegetable oils, such as propylene glycol, polyethylene glycol and olive oil, and injectable ester such as ethyl oleate.

The inventive cell therapeutic agent for inducing hair growth, the inventive agent for treating alopecia and the inventive agent for treating atrichosis may be applied to the scalp in order to treat typical male-type alopecia, which is recognized as baldness, or female-type alopecia which can occur after menopause or oophorectomy. In addition, these agents may be applied to any area of the body, which is in need of hair growth. For example, these agents may be applied to a hair loss site resulting from a traumatic scar, or to a broad forehead or an M-shaped forehead in order to provide a simple beauty effect, and may be used to improve the conditions of atrichosis of eyelashes or eyebrows.

The inventive cell therapeutic agent for inducing hair growth, the inventive agent for treating alopecia and the inventive agent for treating atrichosis may be formulated with a pharmaceutically acceptable carrier. Examples of the pharmaceutically acceptable carrier include, but are not limited to, lactose, dextrose, sucrose, sorbitol, mannitol, starch, rubber arable, potassium phosphate, arginate, gelatin, potassium silicate, microcrystalline cellulose, polyvinylpyrrolidone, cellulose, water, syrups, methylcellulose, methylhydroxy benzoate, propylhydroxy benzoate, talc, magnesium stearate, and mineral oil.

In addition to the above components, the inventive cell therapeutic agent for inducing hair growth, the inventive agent for treating alopecia and the inventive agent for treating atrichosis may further comprise lubricants, wetting agents, emulsifiers, suspending agents, preservatives, and the like. The pharmaceutically acceptable carrier and formulation suitable for the inventive cell therapeutic agent for inducing hair growth, the inventive agent for treating alopecia and the inventive agent for treating atrichosis is described in detail in Remington's Pharmaceutical Sciences (19th^{ed}., 1995).

The inventive cell therapeutic agent for inducing hair growth, the inventive agent for treating alopecia and the inventive agent for treating atrichosis may be formulated with pharmaceutically acceptable carriers and/or excipients according to conventional techniques known to those skilled in the art and may be provided in the form of a unit dose form and a multi-dose form. These agents may further contain a dispersant or a stabilizer.

The inventive cell therapeutic agent for inducing hair growth, the inventive agent for treating alopecia and the inventive agent for treating atrichosis may be used alone or in combination with other conventional drug or surgical therapies for inducing hair growth. Such combination therapy can show maximized efficacy.

For humans, the cell therapeutic agent of the present invention may conventionally be administered once or several times at a dose of 10⁴-10¹⁰ cells/body, and preferably 10⁶-10⁸ cells/body. Particularly, the composition of the present invention preferably contains adult stem cells at a concentration of 1×10⁸ cells/100 μl to 1×10⁹ cells/100 μl.

However, it is to be understood that the actual dose of the active ingredient of the composition should be determined according to various related factors, including the disease to be treated, the route of administration, the patient's age, sex and weight, and the severity of disease. Thus, the above dose does not limit the scope of the present invention in any way.

### EXAMPLES

Hereinafter, the present invention will be described in further detail with reference to examples. It will be obvious to those skilled in the art that these examples are illustrative purposes only and are not to be construed to limit the scope of the present invention.

The sources of the media and reagents used in Examples below are shown in Table 1 below.

TABLE 1

| Items | | Brand | Cat No. |
|---|---|---|---|
| Basal Media and | Media 199 | Gibco | 11150 |
| additives | F-12 Nutrient | Gibco | 11765 |
| | mixture(Ham) | | |
| | EGF | Gibco | 13247-051 |
| | | Miltenyi Biotech | 130-093-827 |
| | bFGF | Gibco | 13256-029 |
| | | Miltenyi Biotech | 130-093-842 |
| | ITS + Premix | BD | 354352 |
| | FBS | Gibco | 16000 |
| | Antibiotic- | Gibco | 15240 |
| | antimycotic | | |
| ROCK inhibitor | Y-27632 | Sigma | Y0503 |
| Tissue | L/G DMEM | Welgene | LM001-11 |
| dissociation | Collagenase type A1 | Gibco | 17100-017 |
| Media | | | |
| Tissue | HBSS | Gibco | 14175 |
| Transprotation | DPBS | Welgene | LB001-02 |
| Buffer solution | Antibiotic- | Gibco | 15240 |
| | antimycotic | | |
| | Normocin | Invivogen | ant-nr-1 |
| Cell dissociation | TrypLE Express | Gibco | 12604 |
| agent | | | |

### Example 1

### Isolation of Follicular Stem Cells

Scalp-derived tissue (Hair Transplantation Center, Korea) was finely cut. The cut tissue was placed in an L/G DMEM medium (Welgene, Korea) containing 2 mg/ml of collagenase type A1 (Gibco, USA) and was chemically degraded in a gravity convection incubator at 100 rpm at 37° C. for 30-50 minutes.

The chemically degraded tissue was collected by centrifugation and washed with DPBS. The washed tissue was cultured in a M199/F12 serum medium (supplemented with a 1:1 mixture of M199 and F12, 0.1×ITS premix, 20 ng/ml of rEGF (Gibco, USA), 10 ng/ml of bFGF (Gibco, USA), 1× antibiotic/antimycotic mixture, and 10% fetal bovine serum (Gibco, USA)). Herein, the 0.1×ITS+ premix contained 0.625 ug/ml of insulin, 0.625 ug/ml of transferrin, 0.625 ug/ml of selenious acid, and 0.535 ug/ml of linoleic acid.

When the tissue stated to adhere to the bottom of the culture dish after about 3 days, the medium was replaced with a serum-free M199/F12 medium (named “P0 cells”). Non-adherent tissue was collected and cultured again in a serum-containing M199/F12 medium (supplemented with a 1:1 mixture of M199 and F12, 0.1×ITS premix, 20 ng/ml of rEGF (Gibco, USA), 10 ng/ml of bFGF (Gibco, USA), 1× antibiotic-antimycotic mixture, and 10% fetal bovine serum (Gibco, USA)) for 3 days, and the cultured tissue was named “P0T1”. Then, the P0 cells were supplied with fresh medium at 2-day intervals, and the medium of the P0T1 cells was replaced with a serum-free M199/F-12 after 3 days. Non-adherent tissue collected from P0T1 was named “P0T2” and cultured again. In the same manner, the P0 and P0T1 tissue/cells were supplied with fresh medium at 2-day intervals, and the medium of the P0T2 cells was replaced with fresh medium after 3 days, and the P0T2 cells were continuously cultured. As a result, follicular stem cells were isolated.

### Example 2

### Efficiencies of Culture of Follicular Stem Cells in Various Media Containing Rock Inhibitor

### 2-1: Culture of Isolated Follicular Epithelial Stem Cells

First, the number and viability of the follicular stem cells obtained in Example 1 were measured. The results of the measurement were as follows:

P0: 1.33×10⁶ (91%)

Then, the follicular stem cells were added to each well of a 12-well plate, and 1 ml of each of control groups and test groups for 9 kinds of media containing ITS+ (insulin, transferrine, selenium) premix, EGF, bFGF and an antibiotic/antimycotic mixture was added to each well of the 12-well plate. 10 μl (10 μM) of the ROCK inhibitor Y-27632 was added to each of the test groups, and the cells were cultured.

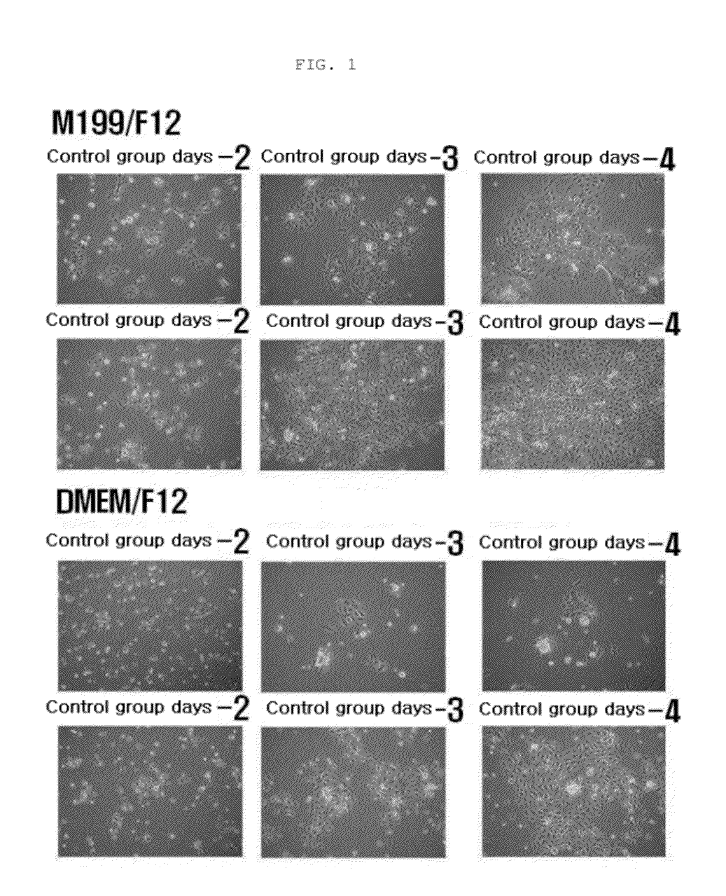

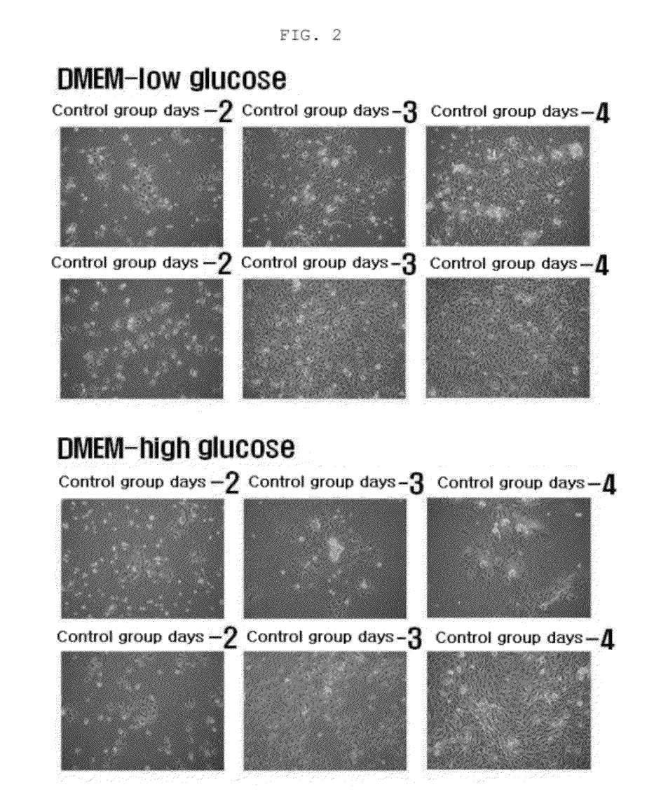

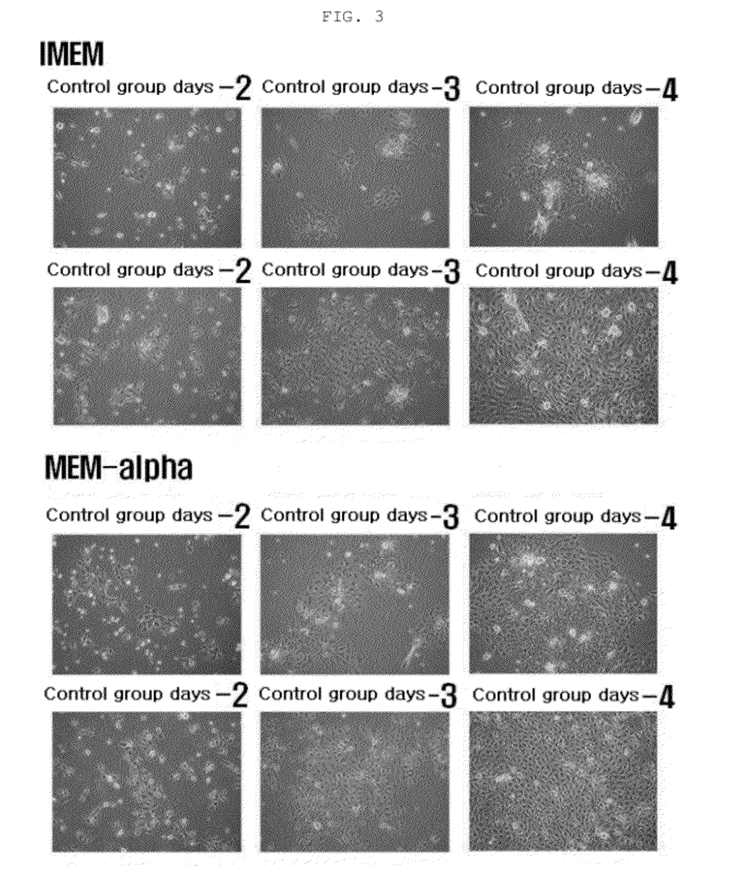

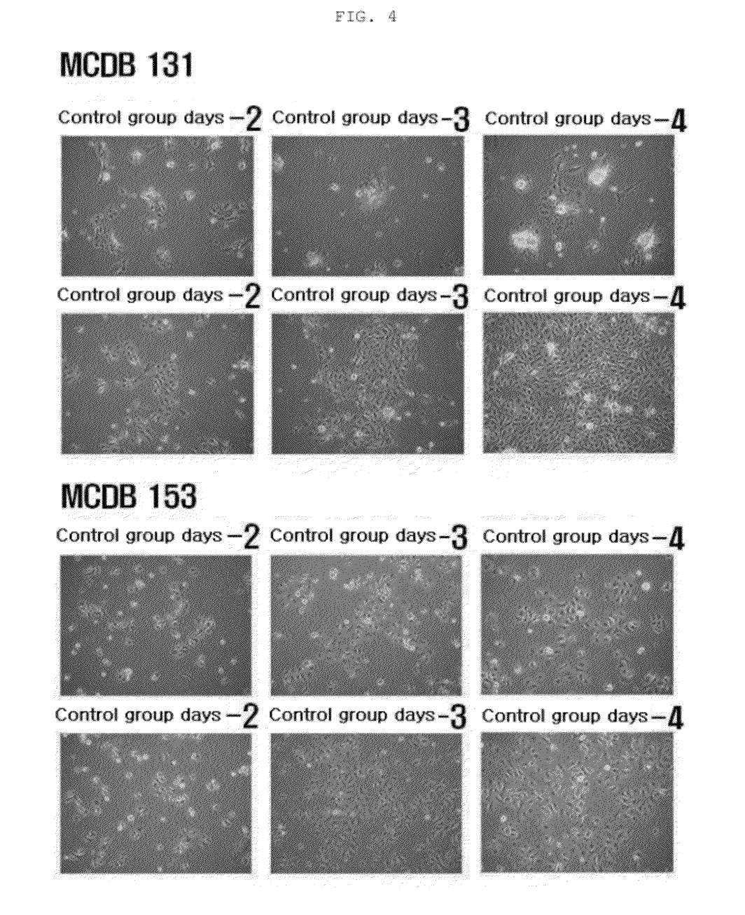

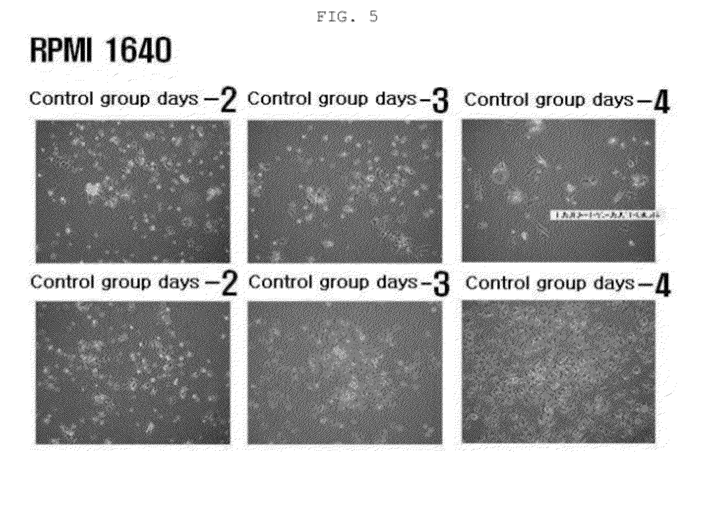

At days 2, 3 and 4 after the start of culture, the state of proliferation of the follicular stem cells in each of the media was observed, and the results of the observation are shown in FIGS. 1 to 5.

TABLE 2

| Nos. | Kind of medium | Manufacturers | Cat. | Lot. |
|---|---|---|---|---|
| 1 | M199/F12 | GIBCO | 11150 | 509175 |
| 2 | DMEM-Low | Welgene | LM001-11 | LM01090211 |
| | glucose | | | |
| 3 | DMEM-High | Welgene | LM001-05 | LM01090405 |
| | glucose | | | |
| 4 | DMEM/F12 | Welgene | LM002-08 | LM02080208 |
| 5 | IMEM | GIBCO | 12440-053 | 444262 |
| 6 | MEM-alpha | GIBCO | 12561 | 548861 |
| 7 | MCDB 131 | Welgene | LM016-03 | LM16080303 |
| 8 | MCDB 153 | Welgene | LM016-05 | LM16080105 |
| 9 | RPMI 1640 | GIBCO | 11875-093 | 571559 |

* supplement: ITS + premix(0.1X), EGF(20 ng/ml), bFGF(10 ng/ml), Antibiotic-Antimycotic(1X)

As can be seen in FIGS. 1 to 5, among the 9 kinds of media, M199/F-12, MEM-alpha, DMEM L/G, DMEM H/G, MCDB 131, IMDM, DMEM/F-12, RPMI 1640 and MCDB 153, the M199/F12 medium, the MEM-alpha medium, the DMEM L/G, the DMEM H/G, the MCDB 131 medium and the IMEM medium allowed the follicular stem cells to proliferate in large amounts.

With respect to the effect of the ROCK inhibitor, the ROCK inhibitor containing M199/F12 medium, MEM-alpha medium, DMEM L/G, DMEM H/G, MCDB 131 medium and IMEM medium had good effects on the proliferation of the follicular stem cells compared to the control groups. Among them, the M199/F12 medium and the MEM-alpha medium showed the most excellent effects.

In other words, it could be seen that the specific media containing the ROCK inhibitor had excellent effects on an increase in the number of follicular stem cells and on the viability of follicular stem cells.

### 2-2: Comparison of the Proliferation Ability of Follicular Stem Cells Between Various Concentrations of Rock Inhibitor

In order to examine the proliferation ability of follicular stem cells at various concentrations of the ROCK inhibitor, the ROCK inhibitor was added at various concentrations to a M199/F-12 medium containing ITS+ premix, EGF, bFGF and antibiotic/antimycotic mixture, and the follicular stem cells obtained in Example 1 were cultured in the medium.

Specifically, 10 μM of the ROCK inhibitor was added to M199/F-12 used in Example 1, and the P1 follicular stem cells were cultured in the M199/F-12 medium and collected by TrypLE-Express treatment. Then, the cells were added to each well of a 6-well plate at a density of 7.70×10⁵ cells, and 2 ml of M199/F-12 was added to each well of the 6-well plate.

The cell suspension was plated in each well of a 6-well plate, and then the ROCK inhibitor Y-27632 was added at concentrations of 10 nM, 100 nM, 1 μM, 10 μM and 100 μM to the 5 wells (excluding the well for a control group) of the 6-well plate, respectively, followed by culture of the follicular stem cells. The medium was replaced at 2-day intervals during culture of the cells, and the cells were photographed 2, 3, 4, 5 and 6 days after the start of cell culture.

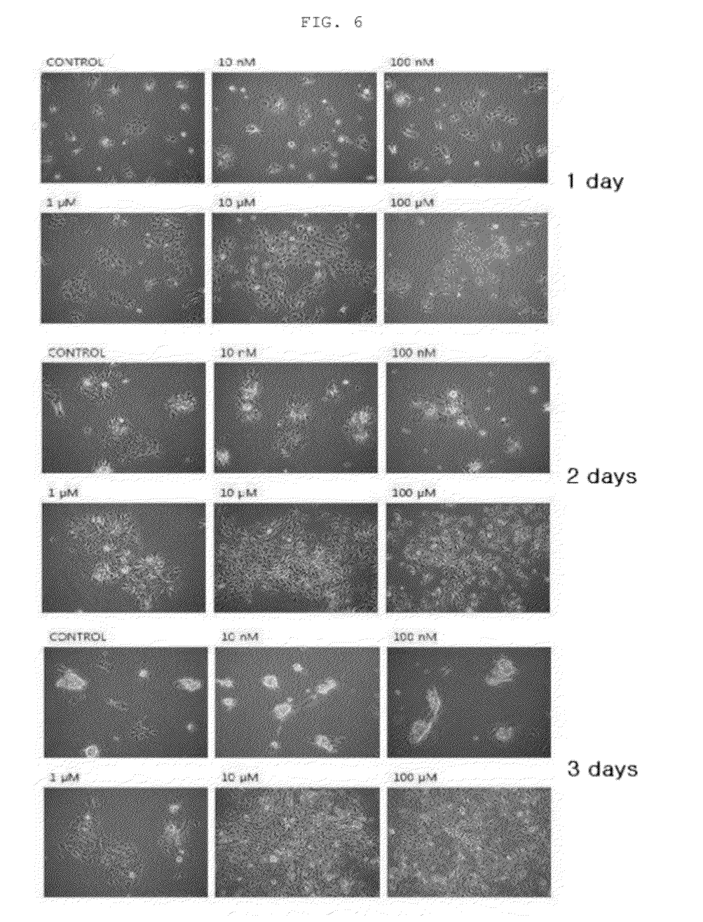

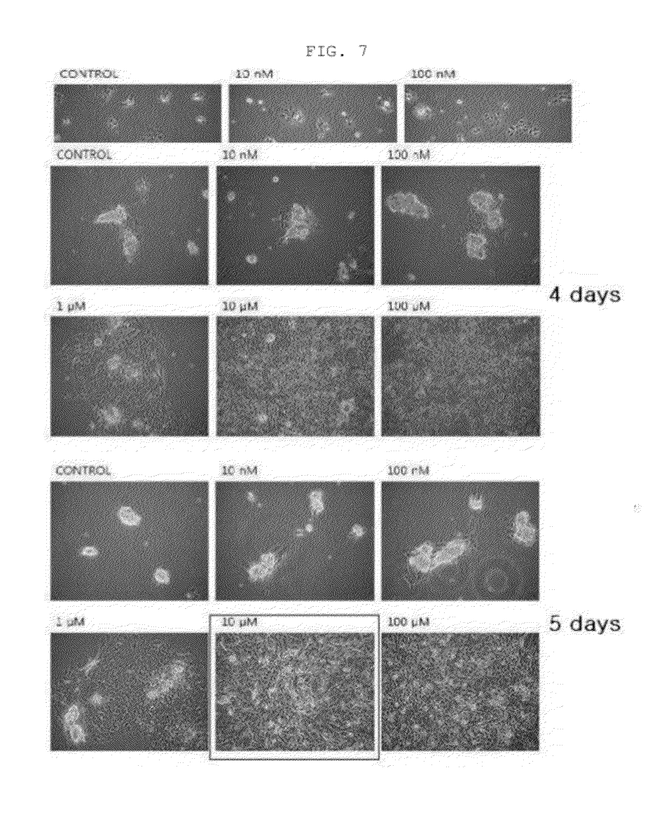

The photographs of the cells are shown in FIGS. 6 and 7.

As can be seen in FIGS. 6 and 7, the proliferation of the cells treated with 100 nM or less of the ROCK inhibitor did not significantly differ from the proliferation of the cells which were not treated with the ROCK inhibitor. In addition, it was observed that the proliferation of the cells was enhanced at a ROCK inhibitor concentration of 1 μM or 100 μM, but the morphological change or differentiation of the cells appeared. Thus, it was determined that the most preferred concentration of the ROCK inhibitor is about 10 μM.

### Example 3

### Determination of Medium Components

In order to find a medium composition suitable for follicular stem cells, the proliferation ability of follicular stem cells was compared between a control group consisting of a commercially available M199/F-12 medium composition alone and a test group consisting of M199/F-12 medium supplemented with ITS+ premix, EGF, bFGF and an antibiotic/antimycotic mixture.

The follicular stem cells obtained in Example 1 were cultured in each of the control group and the test group, and the state of proliferation of the cells was observed 4 days after the start of cell culture.

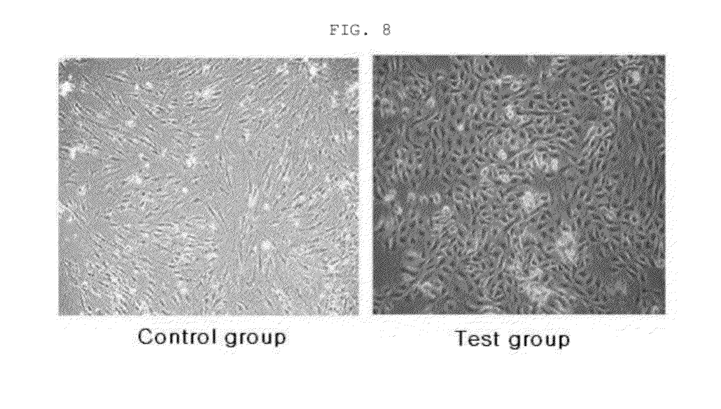

The results of the observation are shown in FIG. 8. As can be seen in FIG. 8, when the follicular stem cells were cultured in the commercial M199/F-12 medium composition alone, the proliferation rate of the follicular stem cells was low and the serious morphological change of the cells occurred. On the other hand, when the follicular stem cells were cultured in the M199/F-12 medium supplemented with ITS+ premix, EGF, bFGF and an antibiotic/antimycotic mixture, the morphology of the follicular stem cells was maintained intact, and the proliferation rate of the cells was significantly high.

The above results suggest that not only the kind of medium, but also specific components which are added to the medium, have a great influence on the proliferation of follicular stem cells.

### Example 4

### In Vivo Examination of the Effect of Stem Cells on the Treatment of Alopecia

### 4-1: Examination of the Effect of Stem Cells on Treatment of Alopecia in Mouse Model

In order to examine whether alopecia is efficiently treated by the follicular stem cells cultured in the present invention, the treatment of alopecia with stem cells was attempted.

The follicular stem cells obtained in Example 1 were administered to androchronogecetic alopecia (B6CBAF1/j) mice as a male-type alopecia animal model. More specifically, dihydrotestosterone or testosterone was injected subcutaneously into 12-week-old female B6CBAF1 hybrid mice (female C57BL/6× male CBA) in an amount of 2 mg/day to induce hair loss, after which each of test materials was administered to the mice.

50 animals used in the test were divided into 5 groups, each consisting of 10 animals.

[Test Groups]

1. Negative control group (saline)

2. Positive control group (3% Minoxidil)

3. Group administered with follicular stem cells (hHFSC) alone

4. Group administered with adipose stem cells (hASC) alone

5. Group administered with a combination of adipose stem cells and follicular stem cells (hASC/hHFSC)

The male hormone was injected subcutaneously into the test animals throughout the test period even after the induction of hair loss.

The condition of hair loss induced in the test animals by treatment with the male hormone dihydrotestosterone or testosterone was evaluated at 2-week intervals using a grading system (graded on a scale from 0 to 4; see Table 3 below).

TABLE 3

| Index | Symptoms |
|---|---|
| 0 | No hair loss |
| 1 | Hair loss in the intrascapular area |
| 2 | Hair loss of 1 cm² or so |
| 3 | Hair loss of 2 cm² or so |
| 4 | Hair loss of 4 cm² or so |

Up to 4 weeks after treatment with the male hormone, a particular hair loss phenomenon could not be observed. From 5 weeks after treatment with the male hormone, in the group treated with dihydrotestosterone, the hair started to become thinner, and downy hair and hair loss events could be observed on the back of the animals. In the group treated with testosterone, the induction of hair loss was slower than that in the dihydrotestosterone-treated group, and hair loss events could be observed from about 7 weeks after treatment.

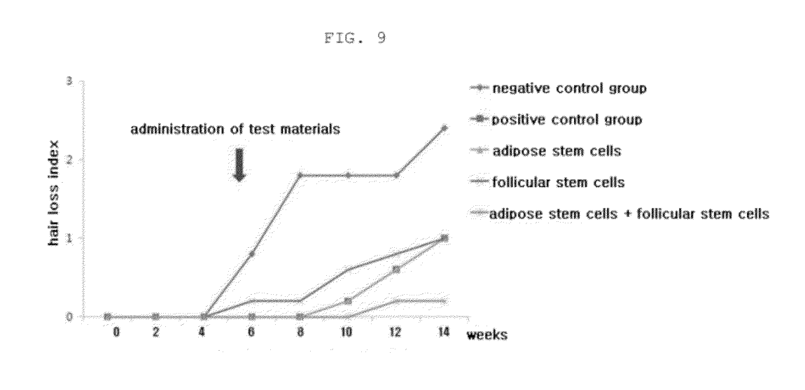

Thus, in the group treated with dihydrotestosterone, the test materials were administered from 7 weeks after treatment, and in the group treated with testosterone, the administration of the test materials was initiated from 9 weeks after treatment. The results of the treatment are shown in FIGS. 9 to 12.

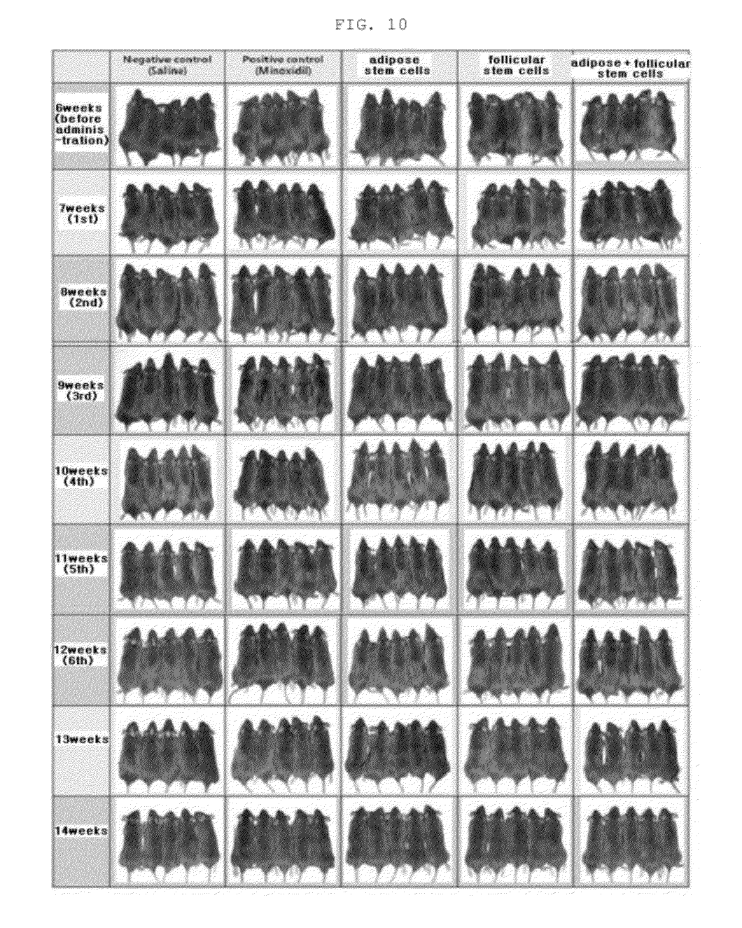

As can be seen in FIGS. 9 and 10, in the group treated with dihydrotestosterone, the condition of hair loss in the group treated with the test material (adipose stem cells alone, follicular stem cells alone or a combination of adipose stem cells and follicular stem cells) or the positive control group administered with Minoxidil started to become significantly better from 1-2 weeks after administration of the test material compared to that in the negative control group. The results of observation conducted 8 weeks after the initiation of administration of the test material indicated that the effect of administration of adipose stem cells alone or follicular stem cells alone on the inhibition of hair loss was equal to that of administration of the positive control (Minoxidil) and that the effect of administration of the adipose stem cell/follicular stem cell combination on the inhibition of hair loss was good compared to those in not only the negative control group, but also the positive control group.

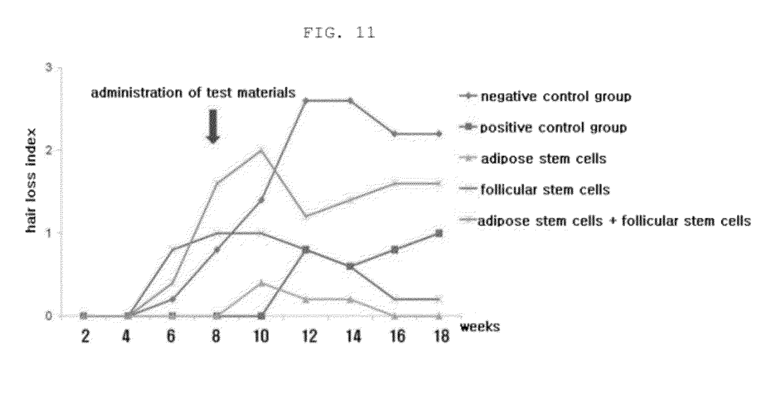

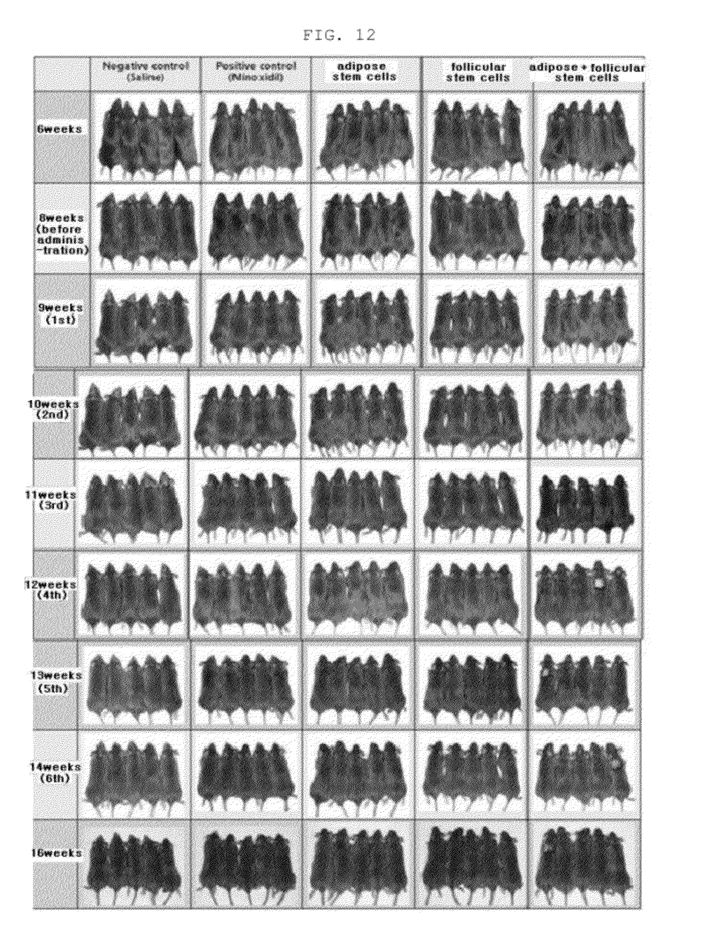

In addition, as can be seen in FIGS. 11 and 12, in the group treated with testosterone, the degree of hair loss in the negative control group started to increase from 2 weeks after the initiation of administration of the test material, whereas the condition of hair loss in the group administered with the test material (adipose stem cells alone, follicular stem cells alone or a combination of adipose stem cells and follicular stem cells) became better. Particularly, at 4 weeks after the initiation of administration of the test material, the progression of hair loss in the group administered with the combination of adipose stem cells and follicular stem cells, which has showed evident hair loss, was significantly inhibited. At 10 weeks after the initiation of administration of the test material, the effects of treatment with adipose stem cells alone and follicular stem cells alone on the inhibition of hair loss was significantly excellent compared to that of the positive control (Minoxidil).

Thus, it was confirmed that, when the follicular stem cells cultured in the medium of Example 1 were used, hair loss diseases were inhibited or treated in a manner similar or superior to those in the positive control group. Such results suggest that the administration of follicular stem cells having an excellent ability to proliferate enables the effective treatment of hair loss diseases.

### 4-2: Examination of the Effect of Combination of Stem Cell on Treatment of Hair Loss

Adipose stem cells derived from adipose tissue are significantly easy to isolate and collect, and methods for proliferating and maintaining these adipose stem cells are widely known. Thus, in order to whether the use of adipose stem cells in combination with follicular stem cells which are not relatively easy to isolate has an effect on the treatment of hair loss diseases, the following test was carried out.

A hair loss disease in mice was induced in the same manner as in Example 4-1, and the effect of the cells on the treatment of hair loss was examined. Dihydrotestosterone was injected subcutaneously into 12-week-old female B6CBAF1 hybrid mice (female C57BL/6× male CBA) in an amount of 2 mg/day for 6 weeks, and as a result, evident hair loss could be observed on both the thighs and the abdominal region.

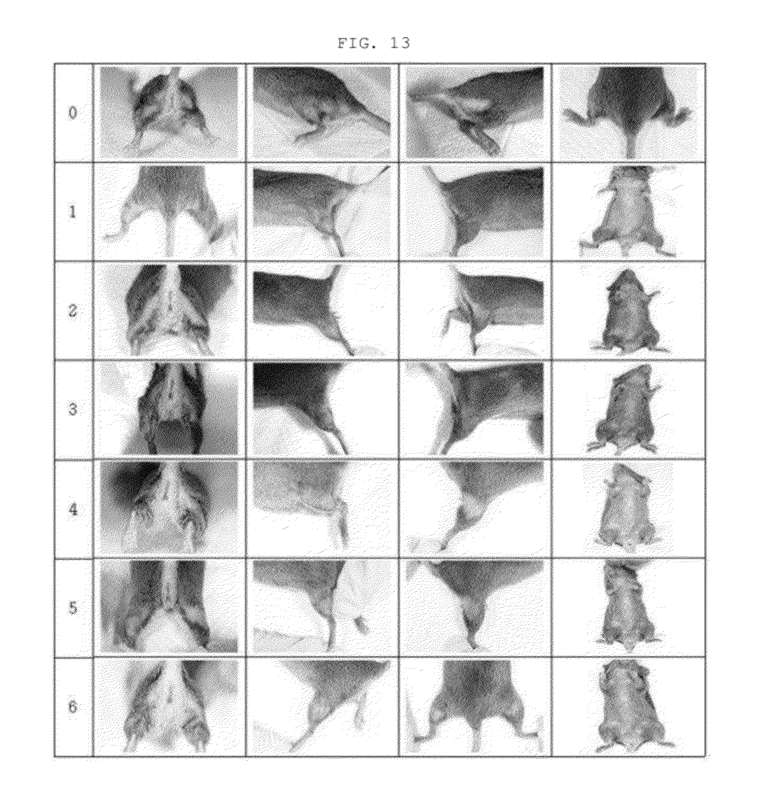

Thus, adipose stem cells and follicular stem cells were alternately administered to the thigh region at 3-4-day intervals (once weekly administration of adipose stem cells+once weekly administration of follicular stem cells) for 6 weeks. During the administration of the test material and the subsequent observation period, the male-type hormone was injected subcutaneously into the test animals daily. The mice administered with the test material for 6 weeks were photographed and the photographs are shown in FIG. 13.

As can be seen in FIG. 13, the results of visual observation conducted 1 week after the initiation of administration of the stem cells indicated that hair grew on the hair loss site on the abdomen. However, the growth of hair on the site administered with the cells or other hair loss sites was insignificant. However, 2 weeks after the initiation of administration of the stem cells, the growth of hair on the hair loss site on the abdomen could be observed, and hair loss on both the thigh sites no longer progressed, indicating that the hair loss disease was treated.

It was confirmed that, in the case of a hair loss disease whose treatment requires a large amount of stem cells, when adipose stem cells which are relatively easy to isolate and collect are used in combination with follicular stem cells, the hair loss disease was inhibited or treated in a manner similar or superior to that in the administration of follicular stem cells alone. Such results suggest that, when adipose stem cells are administered in combination with follicular stem cells having an excellent ability to proliferate, the effective treatment of hair loss diseases is possible.

Although the present invention has been described in detail with reference to the specific features, it will be apparent to those skilled in the art that this description is only for a preferred embodiment and does not limit the scope of the present invention. Thus, the substantial scope of the present invention will be defined by the appended claims and equivalents thereof.

### INDUSTRIAL APPLICABILITY

The inventive medium for culturing follicular stem cells enables follicular stem cells isolated from scalp tissue to proliferate in such a large amount that the follicular stem cells are clinically applicable. Thus, the follicular stem cells will be useful as cell therapeutic agents for hair growth for hair loss diseases such as alopecia and atrichosis.

1. A medium for proliferating and maintaining follicular stem cells, which contains 5-50 μM of a Rho-associated kinase (ROCK) inhibitor in a basal medium.

2. The medium of claim 1, wherein the basal medium further contains an ITS+ premix (insulin, transferrin, selenious acid premix), EGF, bFGF, or antibiotics.

3. The medium of claim 1, wherein the Rho-associated kinase (ROCK) inhibitor is contained in a concentration of 5-20 μM.

4. The medium of claim 1, wherein the Rho-associated kinase (ROCK) inhibitor is contained in a concentration of 10 μM.

5. The medium of claim 1, wherein the basal medium is at least one selected from the group consisting of an M199/F12 (mixture) medium, a MEM-alpha medium, a low-concentration glucose-containing DMEM medium, a MCDB 131 medium, and an IMEM medium.

6. The medium of claim 1, wherein the basal medium is either an M199/F12 (mixture) medium or a MEM-alpha medium.

7. The medium of claim 1, wherein the follicular stem cells are derived from human scalp tissues.

8. The medium of claim 1, wherein the Rho-associated kinase (ROCK) inhibitor is a compound having a structure represented by the following formula 1:

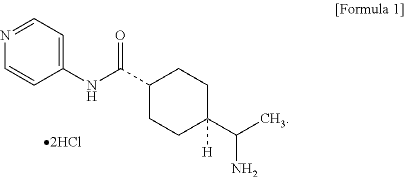

9. A method for proliferating and maintaining follicular stem cells, the method comprising subculturing isolated follicular stem cells in the medium of claim 1.

10. The method of claim 9, wherein the isolated follicular stem cells are subcultured to passage 2-6.

11. A composition for treating hair loss, which contains as an active ingredient follicular stem cells obtained by the method of claim 9.

12. The composition of claim 11, wherein the composition for treating hair loss further contains adipose tissue-derived stem cells.
